(19) United States
(12) Patent Application Publication
(10) Pub. No.: US 2006/0216193 A1
(43) Pub. Date: Sep. 28, 2006

(54) Cleaning tools with UV flash unit

(76) Inventors: Kaj A. Johnson, Oakland, CA (US); Douglas J. Minkler, Oakland, CA (US); Scott L. Cumberland, Pleasanton, CA (US); Joyce Delosreyes, Pleasanton, CA (US); Steven E. Bromberg, Pleasanton, CA (US); Edward B. Rinker, Pleasanton, CA (US); Aram Garabedan JR., Pleasanton, CA (US); Ricardo Ruiz De Gopegui, Pleasanton, CA (US)

Correspondence Address: THE CLOROX COMPANY, P.O. BOX 24305, OAKLAND, CA, 94623-1305, US

(21) Appl. No.: 11/086,021

(22) Filed: Mar. 22, 2005

(51) Int. Cl. A61L 2/00; G01J 3/10; A61N 5/06; H05G 2/00

(52) U.S. Cl. 422/24; 250/504 R

(57) ABSTRACT

A combination tool for cleaning and sanitizing a surface is disclosed. The tool includes a cleaning device and at least one UV flash unit. The cleaning device can be adapted for either wet or dry cleaning. The combination tool can also include a dispensing unit that can dispense a treatment agent. The tool can also include a sensor that can detect surface properties. In some arrangements, the tool includes a motive component that can propel the tool robotically.

### BACKGROUND OF THE INVENTION

1. Field of the Invention

This invention relates generally to cleaning tools that can destroy microorganisms and pathogens, and, more specifically, to portable cleaning tools that use low power UV radiation.

2. Description of the Related Art

UV light can be used in disinfection processes to destroy microorganisms, and pathogens, such as bacteria, bacterial spores, viruses, mycoplasma, protozoans, oocysts, and toxins, but typically must be delivered with enormous fluence and exposure (absorbed energy per unit area). See, for example, Clark et al., U.S. Pat. Nos. 5,786,598 and 5,925,885, Dunn, U.S. Pat. No. 4,871,559, Hiramoto, U.S. Pat. No. 4,464,336, and Busnell, U.S. Pat. No. 5,768,853, all of which are incorporated by reference herein. Multiple UV light sources and long exposure times are not practical for many applications such as decontamination or disinfection of people, equipment, and spaces that must be returned to activity or use as quickly as possible. Although more powerful light sources can reduce exposure times, they are not practical; long exposure to UV light can cause degradation of many materials and harmful biological effects such as erythema and burns. Higher average power and higher fluence sources generally have lower efficiencies, increased power consumption which limits portability, and excess heat generation.

There are many additional situations in which methods and apparatus for UV disinfection, which can be used instantly as needed, would be beneficial. Applications for such a system include cleaning and disinfection of surfaces in homes, medical, food preparation, and pharmaceutical facilities. There are many additional situations in which benefits could be realized by methods and apparatus that can be rapidly deployed or used on an occasional basis or in variable environmental conditions.

In the past, continuous UV sources used for disinfection have been mainly quartz tubes filled with mercury vapor. Continuous UV sources are generally heavy and cumbersome and require high power. With the advent of low power UV sources, such as xenon flash lamps and UV light-emitting diodes (LEDs), which are lightweight, compact, and require much less power, possibilities for incorporating UV sources into portable cleaning tools have multiplied.

### DETAILED DESCRIPTION

The terms “light” and “UV light” are used herein to mean electromagnetic radiation that includes at least a portion in the spectrum of wavelengths associated with UV radiation (˜1-400 nm) and may or may not include portions in the visible band (˜400-700 nm) and in the infrared band (˜700-10⁵ nm). The UV spectrum can be further subdivided into three smaller bands: UVA (˜320-400 nm), UVB (˜290-320 nm), UVC (˜100-290 nm). The term “visible light” is used herein to mean electromagnetic radiation in the band of wavelengths associated with visible radiation (˜400-700 nm).

The term “wet cleaning” includes delivering active liquids to surfaces such as walls, counters, floors, carpets furniture, drapes, interior wall spaces, conduits, ventilation ducts, pipes, etc. for the purpose of removing undesirable materials such as dirt, allergens, microorganisms, etc. The active fluids can be delivered in any number of ways, such as through pouring from bottles, sprays, aerosols, foggers, hoses, misters, breakable capsules/(e.g. dry liquids), or wet wipes.

The term “dry cleaning” includes removing undesirable materials such as dirt, allergens, microorganisms, etc. from surfaces such as walls, counters, floors, carpets furniture, drapes, interior wall spaces, conduits/ventilation/pipes, etc. without the intentional application of liquids. Dry cleaning can involve wiping with a dry cloth or non-woven or it can involve vacuum cleaning. Active vapors or solids (e.g., powders, nanoparticles) can be used to aid in dry cleaning. Dry cleaning can involve pre-application of liquids that dry quickly to become integral with the surface and then subsequent removal of the dry residue.

The terms “denature,” “disinfect,” and “sanitize” are used herein to mean neutralize or inactivate biological contaminants. Biological contaminants can include microorganisms, mites, fungi, mold, mildew, dust mites, fleas, insect eggs, and allergens that can cause an adverse reaction in people or animals. The term “treatment” is used herein to mean denature, disinfect, sanitize and/or deodorize.

The term “surface” is used herein to mean the material layer or layers at the outermost boundary of an object. Surfaces of different objects can have different depths. For example, a smooth, painted plaster wall has a thin surface which includes irregularities—both high regions and low regions due to the texture of both the paint and the plaster. The surface can extend to a depth within the wall where dirt and contaminants can collect. In another example, a deep plush carpet has a thick surface that includes the entire length of the carpet fibers and the side of the backing to which the fibers attach.

Continuous ultraviolet light disinfection devices, which use mercury vapor lamps that emit in a single germicidal wavelength (254 nm), have been used for more than 50 years to disinfect surfaces and fluids. Mercury vapor lamps consume substantial energy (input power ranges up to 300 watts) and have efficiencies less than about 40%, with the best efficiencies at operating temperatures of about 40° C. Mercury vapor lamps are predominantly AC powered because of long exposure requirements for germicidal effectiveness. The energy requirement alone makes most portable applications impractical for mercury lamp UV systems, as connection to power cords limits mobility.

A mercury vapor lamp is a sealed glass tube with an electrode at each end and is filled with an inert gas (usually argon) and small amounts of liquid mercury. The tube is usually made of quartz. In many applications, a double-walled tube is used as a safeguard against release of toxic mercury if the outer tube wall is breached. Mercury vapor lamps are heavy and expensive.

A mercury vapor lamp is typically left on during UV processing so that the user does not have to wait through the long warm-up period required each time the lamp is turned on. Often a mechanical shutter is used during the non-irradiation phases (the “off” periods) of a process to block the light. Mechanical shutters can jam, presenting safety and process flexibility issues and causing difficulties with using the light efficiently.

Pulsed or flash UV light units are significantly different from continuous UV light units. The term “flash” is used to describe an ultraviolet light source that is not operated continuously for longer than about 1 second. A single flash lasting less than 200 microseconds from a xenon flash lamp can output many times more UVC radiation than a comparable mercury vapor lamp can output over many minutes. A xenon flash lamp emits a broad spectrum of germicidal wavelengths in the 200-310 nm range and is more effective than a mercury vapor lamp per unit of energy.

Germicidal ultraviolet flash technology provides rapid, effective, low-cost solutions for a broad array of disinfection and sterilization applications. Germicidal UV is effective against bacteria, viruses, molds, spores, odor, and fungi and can prevent the colonization and transmission of microorganisms that cause infections or allergic reactions. Xenon pulsed UV lamps can be customized in a wide variety of shapes and sizes to meet specific requirements. Example shapes include cylinders, rectangles, circles, and helices. UV flash lamps for sterilization/disinfection have been described by UV Solutions in U.S. Pat. Nos. 6,730,113; 6,461,568; and in U.S. Patent Application Publication Nos. 2004/0034398A1, 2003/0031586A1, 2003/0018373A1, and 2003/0017073A1, all of which are included by reference herein.

Pulsed UV offers ease of operation. Typical turn-on times are in the range of 1 to 5 microseconds; virtually no warm-up time is required. Instant on/off control with a pulsed UV light unit results in expending energy to produce light only when desired. The low operating temperature of pulsed UV light units prevents undesired effects from rapid heating, such as, for example, burning of surfaces. Although some shielding is still desirable, unwanted radiation exposure is less likely to occur because the flash unit is on only when it is being used.

Power levels with pulsed light can be as high as 1×10⁶ watts, with pulse times in microseconds. A UV flash lamp spectrum is also directly dependent on electrical operating conditions; changing the electrical operating conditions can change the peak wavelength. A broad UV spectrum can be tuned to peak in particular spectral ranges as needed for specific applications.

UV light from a pulsed flash lamp unit can penetrate into a surface with sufficient energy for sterilization and/or molecular-bond dissociation. In comparison, a continuous, mercury vapor lamp delivers much of its energy to a surface as heat. Although a flash lamp can emit UV light at very high power, a relatively small amount of energy is required due to the short pulse duration. The same amount of energy can be used to power either a 10-watt continuous mercury vapor lamp for 10 seconds or a 10,000,000-watt pulsed lamp for 10 microseconds.

A UV transparent window, made of a material such as quartz, fused silica, or UV transparent glass or plastic, may be included to protect the flash lamp from mechanical damage while maximizing the output of UV light in a pulse. The window can include an optical filter to alter the spectrum of the emitted light, which can result in an optimized spectrum on a treatment surface, a spectrum having greater efficacy and/or having a more benign effect on the treatment surface itself. The window can contain a lens element that provides focus or dispersion of the UV light or is configured to provide a desired shape to the UV light stream. The window can include a textured surface or other diffusing mechanism to alter the exit angle of the emitted light and thereby reduce shadowing on an irregular treatment surface.

A UV flash lamp circuit can contain a capacitor or array of capacitors. The capacitor(s) can store charge from a power source and discharge subsequently on command to power a flash of UV light. An array of capacitors can be arranged so that the capacitors discharge in a continuous sequence to maintain continuous UV flashing. In some arrangements, a UV flash unit is cordless, getting power from an onboard battery or set of batteries. The battery(ies) may or may not be rechargeable. Typically several hundred or more sterilization/disinfection operations can be performed using a single set of batteries. In other arrangements, external power from an AC power source can be used in addition to batteries or instead of batteries. In other arrangements, fuel cells or solar cells can be used to meet some or all of the energy requirements.

In one arrangement, an array of batteries with suitable discharge characteristics can provide power to the UV flash lamp directly without an intermediate capacitor. For example, Altair Nanotechnologies of Reno, Nev., has produced a nano-sized lithium titanate spinel battery material that exhibits charge and discharge rates up to 100 times higher than materials used in current commercially available batteries (“Altair Advances Materials,” Battery & EV Technology 29 (4), 9, (2004)). Sides et al have reported nanostructured electrodes that show dramatically improved discharge rates compared to conventional thin-film electrodes for lithium-ion batteries (“Nanoscale Materials for Lithium-Ion Batteries,” MRS Bulletin, August 2002, pp. 604-607).

Advantages of pulsed UV light units over continuous UV light units include faster on/off processing, low energy requirements (obviating the need for power cord connections to household electrical systems), small, compact size, low weight, non-toxic materials, and the possibility for portability that results from these attributes. In addition, pulsed UV light can operate at wavelengths and intensities that can penetrate opaque material. Yet, because of the short duration of the pulses, there is little or no surface temperature buildup. Units that produce pulsed UV light do not contain toxic chemicals or materials, such as mercury. Pulsed UV light is particularly useful in meeting safety and flexibility requirements and in applications where continuous light raises the temperature of the surroundings or is unable to penetrate opaque materials. Pulsed UV units use much less energy and are smaller and more lightweight than continuous mercury vapor UV lamps. Pulsed UV is ideal for portable applications. Heretofore it has been difficult to conceive of a UV cleaning tool that was small and light enough for hand-held or automated applications. Flash UV units open many possibilities because their small size, light weight, and ability to have self-contained power supplies make them portable and user friendly, unlike continuous UV lamps.

As discussed above, pulse or flash duration is less than about −1 second in flash UV units. In some embodiments, the flash duration is less than about 0.5 seconds. In other embodiments, the flash duration is less than about 100 milliseconds. In yet other embodiments, the flash duration is less than about 100 microseconds.

UV light can also be produced by semiconductor optical devices, such as light-emitting diodes (LEDs) and lasers, that emit light in the UV range. UV LEDs and their potential uses have been discussed in news releases from Sandia Laboratories (http://www.sandia.gov/news-center/news-releases/2003/elect-semi-sensors/uvleds.html and http://www.sandia.gov/news-center/news-releases/2003/elect-semi-sensors/uvuse.html). Some UV LEDs are made with a sapphire substrate and conductive layers of the semiconductor aluminum gallium nitride. The wavelength output by UV LEDs can be changed by adjusting the amount of aluminum in the semiconductor layers. For example, a mix with about 50% aluminum produces UV light with a wavelength of about 275 nm. UV LEDs are very small—no larger than several millimeters in size—and can be arranged in arrays with any geometry. UV LEDs can add UV disinfection functionality to very compact tools without making any significant increase in the size of the tools. UV LEDs can operate as both pulsed and continuous UV units. Both modes of operation are very energy efficient and use much less energy than continuous mercury vapor lamps. Embodiments of the invention discussed below for UV flash units (both xenon flash lamps and UV LEDs in flash mode) can also be carried out using UV LEDs in continuous mode. A UV LED array can be powered directly with conventional commercial batteries.

A UV flash unit can be small, compact, lightweight and can have a self-contained power source such as a battery. The UV flash unit can be incorporated into implements for large-scale treatment, such as for floors. The UV flash unit can be incorporated into an implement such as a wand, pen, or gun for treatment of small spaces. Optical fibers can be used to guide the UV light to exit from a thin tip in the implement. The thin tip can be inserted into crevices and small spaces to denature, sanitize and/or disinfect (treat) areas that are difficult to clean in other ways. In one embodiment, treatment can be achieved by sweeping the implement back and forth over an area of interest. The cycling of the flash UV unit (repeatedly turning the flash unit on and off with a desired flash duration and frequency of occurrence) can be adjusted for various sweep speeds and for various surfaces (e.g., hard countertop, plush carpet) to ensure that a desired treatment is effected.

In one embodiment of the invention, a combination tool for cleaning and sanitizing a surface includes both a cleaning device and a UV flash unit. The combination tool is compact, portable, light weight, can be powered by batteries, and is thus self-contained. The UV flash unit can be adjacent the working part of the cleaning device, such as a mop head or a suction opening of a vacuum cleaner. Thus the disinfecting action of the UV unit can be utilized wherever cleaning is performed. Biological contaminants on the surface are exposed directly to the disinfecting action of the UV flash unit where they lie. Examples of surfaces include floors, carpets, curtains, upholstery, countertops, windows, walls, ceilings, bathroom surfaces, outdoor surfaces, automobile exteriors and interiors, and patios. The exposure can occur repeatedly, every time a surface is treated by the combination tool. It may take only one exposure to reduce the number of biological contaminants, such as microorganisms, fungi, allergens, and the like, practically to a state of sterility or neutrality. Repeated exposure increases the likelihood that the state of sterility or neutrality will persist.

The embodiments of the combination tool described herein can be suitably designed to include devices for wet cleaning, dry cleaning, or both. Examples of cleaning devices that have apparatus to effect dry cleaning include dry mops, dusters, vacuum cleaners, various vacuum cleaner nozzles, such as combined suction and electrically-powered beater nozzles, upholstery nozzles, curtain nozzles, and the like. Examples of cleaning devices that have apparatus to effect wet cleaning include wet mops, mops with removable pads, scrubbers, brushes and the like.

The UV flash unit of the combination tool can have any of a variety of configurations. In one arrangement, UV light is channeled through optical fibers to expose only small regions of the surface, such as cracks or crevices. In one example, optical fibers are incorporated into vacuum cleaner brushes that can comb through a carpet to liberate dirt and to deliver UV light deep into the carpet. In another arrangement, UV light is shined broadly over a surface. The UV light can be emitted broadly from the configuration of the flash unit(s) or can pass through a diffusing material before reaching the surface. In some arrangements, large combination tools can weigh between about 1 and 15 kilograms. In other arrangements, large combination tools can weigh between about 5 and 10 kilograms.

In some embodiments, the combination tool includes in the cleaning device various other apparatus, such as squeegees, scrubbers, rollers, slides, flat surfaces, surfaces having patterned high spots and low spots, irregular surfaces, brushes, and pads, which can be used alone or in conjunction with a treatment agent. In some arrangements, the cleaning device includes an electric motor to facilitate cleaning of a surface. Power sources used by the UV flash unit can also be used by the cleaning device. The electric motor can cause devices such as brushes, scrubbers, rollers or even the cleaning device or the entire combination tool to move in a lateral or rotational pattern or a random motion. Further discussion of suitable cleaning devices can be found in U.S. Patent Application Publication Nos. 2004/0141798 and 2004/0184867, both of which are incorporated by reference herein.

In other embodiments, combination tools are very small and can be used for detail cleaning. The UV flash unit is incorporated into an implement such as a wand, pen, or gun that includes a small wet cleaning device, such as a brush or wipe, or a small dry cleaning device, such as a small dusting attachment or a vacuum nozzle. Small combination tools can also include a spray bottle or small container containing a treatment agent that can be dispensed to aid in cleaning or in UV treatment. Small combination tools can be very light in weight. In one arrangement, a small tool weighs between about 25 and 100 grams. Intermediate sized combination tools can weigh between about 100 grams and 1 kilogram.

In one embodiment, a combination tool is swept across a surface manually. As a user moves the tool forward and backward, the tool engages a mechanism that can convert some of the motion into electrical energy and store the energy in an energy storage unit. In one example, as the tool is moved by the user in the forward direction, only the cleaning device is active; the treatment unit is inactive and no UV light is emitted. During backward motion, the cleaning device of the tool may or may not be active, and the UV unit is activated using energy from the energy storage unit and, in some arrangements, from a battery as well. In both the backward and forward directions, the tool acts as a manual generator, converting some of the motion into electrical energy and storing it in the energy storage unit.

The combination tool can also include a motive component to propel the tool. In some arrangements, a user can activate the motive component to aid in moving the tool over the surface to be cleaned. In other arrangements, the motive component can propel the tool robotically, that is, the motive component operates automatically or by remote control using one or more sets of pre-programmed instructions. In yet other arrangements, the motive component can operate semi-automatically, following pre-programmed instructions subject to user modifications or interruptions. Methods and systems for controlling robotic cleaning tools have been described in U.S. Pat. Nos. 6,845,297, 6,809,490, 6,781,338, and 6,690,134, all assigned to iRobot Corporation and all of which are included by reference herein.

UV radiation dose is defined as the product of UV intensity (energy per unit surface area) and duration of exposure. The UV radiation dose required to sterilize a surface is depends on the surface properties and the environmental conditions. For example, a total exposure of about 10 milliwatt-seconds/cm² is enough to disinfect most surfaces of a smalls objects.

A UV control system can vary the UV flash intensity and duration (dose), energy (wavelength), and/or time interval between flashes. The UV light dose may be varied as desired to adjust UV light penetration for individual surfaces. For example, UV dose may be higher for deep penetration into a dense pile carpet than for merely penetrating the very top surface of a smooth, hard floor. The UV dosage control can be continuously variable or variable in discrete steps as adjusted by a user. In one embodiment, a user can set the intensity of UV radiation delivered by the UV flash unit to achieve a specific level of treatment. For example, when using a combination tool with a UV flash unit for the first time or for maximum UV treatment where contaminants are a particular problem, a setting that yields very intense UV radiation and therefore effects a very strong treatment can be used. In areas where UV flash is used regularly, a lower dose UV radiation setting can be used to control the growth and spread of any additional contaminants that have appeared since the last cleaning. A low dose setting can help to conserve electric power when a high dose setting is deemed unnecessary. The control system can include a microcontroller or a logic device. The control system can monitor the battery(ies) in the tool and notify a user when the energy level is low. In some arrangements, the control system can give a user a choice as to how to use remaining energy, e.g., whether to decrease the UV treatment dose or to suspend power to the cleaning device or motive component of the tool (in embodiments where the cleaning device uses power and/or there is a motive component that uses power).

In one embodiment, UV light can be directed into a chamber within the tool where debris that has been picked up by the cleaning device is collected. This feature can provide additional disinfection to dirt and debris that has been collected in the chamber by the tool and/or to the inside of the chamber itself. In another embodiment, UV light can be directed into an air exhaust stream before it leaves the tool, to ensure that contaminants are not re-released. In yet another embodiment, a combination tool that includes a vacuum cleaner can be run as an air purifier when it is not being used for cleaning. The tool can pull in air and treat the air with the UV unit before allowing the air to exit the tool in the exhaust stream. Filters, such as carbon filters or HEPA filters can also be used to remove particulate contamination.

For robotically controlled treatment units, the control system can manage both the UV flash unit and movement of the unit. UV dose can be adjusted to be specifically tuned for the rate of travel of the tool, thus avoiding too much or too little UV treatment. The preprogrammed instructions can ensure that the tool covers all accessible regions of the surface to be cleaned at least once. Robotically-controlled units can clean very large surfaces, such as floors, automatically. Sensors that determine properties of the surface to be treated can be particularly useful for a robotic tool, as the tool can move over a large area and can encounter various surfaces, e.g., carpet and hard flooring.

For example, in the case of a UV flash unit as part of a robotically propelled combination tool, the flash cycling (UV intensity and on/off periods) can be tied to the speed of the robot's motion. In some arrangements, the flash cycling can automatically adjust with changes in the robot's speed, start/stop events, maneuvering, or changes in direction. In one example, a robotic vacuum cleaner automatically adjusts the height of the suction opening or the height of floor brushes when it moves from a hard floor surface to a carpeted surface. A feedback mechanism can inform the UV flash unit of the surface change and the flash cycling is adjusted accordingly. In another example, a flash UV system in a manual cleaning tool has its flash cycling set to correspond to an “average” sweep rate for the tool. In one arrangement, the cycling is preset by the manufacturer. In another arrangement, the UV system receives information about the tool speed from a sensor or feedback mechanism and can adjust the flash cycling accordingly. In some arrangements, a user can adjust the flash cycling while using the tool to deliver more UV radiation (greater dose) per unit time when sweeping the tool more quickly or less UV radiation (less dose) per unit time when sweeping the tool less quickly in order to deliver about the same dose to each portion of the surface independent of the tool speed. A user may also want to adjust the dose in response to the levels of contamination encountered during cleaning. For example, a user may want to use a higher UV dose when cleaning near the family dog's favorite area on the carpet than when cleaning a hard floor in a room that is used seldom.

In one embodiment, the control system of the tool senses when the energy supply is low and can instruct the motive component to return the tool to a docking station where the energy storage unit can be recharged. At the docking station other tasks can also be performed, such as replenishing cleaning consumables and/or offloading collected cleaning waste. In some arrangements, the control system can also monitor the level of cleaning consumables and/or collected cleaning waste and instruct the motive component to return the tool to the docking station when replenishing and/or offloading, respectively, is warranted.

In some arrangements, the tool can include some means to determine properties of the surface to be treated. Information of interest can include surface depth, color, surface type, hardness, texture, moisture level, surface roughness, odor, reflectivity, and extent of dirt and/or contaminants. Sensors, such as optical sensors, vibration or piezoelectric sensor, traction sensors, air flow sensors, contact sensors, electronic noses, and near infrared moisture sensors, can be used to gather the information. In one arrangement, a feedback mechanism from a motive component can give an indication of the surface properties; more torque is needed to move the tool through a deep carpet than across a hard floor.

Information from the sensor(s) can be communicated to the control system and used in a pre-programmed algorithm to adjust UV intensity to be specifically tuned for the surface being treated. For example, when the reflectivity of a surface is high, much of the UV light does not penetrate the surface. For hard surfaces, it may be useful to move the tool close to the surface and reduce the UV intensity. For soft pile carpet, it may be useful to move the tool less close to the surface and increase the intensity of the UV or dispense treatment agents that will enhance the effectiveness of the UV treatment, as will be discussed below.

A UV unit can include a UV light seal disposed along the perimeter of a treatment or UV flash unit or along the perimeter of a combination tool and made to make contact with a surface. When the UV light seal is pressed against the surface, the seal prevents escape outside of the treatment area of any UV light emitted by the flash unit and thus prevents injury or discomfort to a user or others nearby. The UV light seal is opaque to UV radiation and may or may not block visible light also. In one embodiment, the UV light seal is formed of a flexible material so that the material can adjust to the contours of an irregular surface and still form a light seal. In some arrangements, the light seal can extend beyond the combination tool bottom perimeter to enclose a larger area than the area covered by the tool footprint.

The UV unit can include safety sensors and interlocks that shut off the UV unit when an unsafe condition is detected. Unsafe conditions include those where UV light is directed away from the treatment surface and in directions where it can irradiate people or animals. A safety indicator may be included to alert a user that safety interlock actuators have been activated, and hence that the UV flash unit may be operated without concern about UV exposure outside the tool. A flash indicator may also be used to indicate that a successful flash has occurred.

The UV unit can contain safety interlock actuators coupled to the UV light seal to prevent accidental activation of the flash unit when the unit is not positioned to prevent UV exposure outside of the exposure chamber. Safety interlock actuators can detect compression of the UV light seal at one or more locations to verify that the seal is placed against a surface before the flash unit can be activated. In some arrangements when the UV light seal is also opaque to visible light, an alternate or additional safety interlock in the form of a photodetector is included to prevent the flash unit from pulsing unless the exposure chamber contains substantially no visible light, indicating that the light seal is in a position to prevent UV exposure outside of the exposure chamber.

In one embodiment of the invention, a treatment agent(s) is included to be used in concert with the combination tool. In some arrangements, a dispenser unit that can dispense the treatment agent(s) is included as part of the combination tool. Treatment agents include gases, liquids and solids (e.g., powders, nanoparticles) that can improve the efficacy of UV treatment. Useful liquid cleaning compositions, antimicrobial, anti-allergen, and miticide agents are disclosed in U.S. Patent Application Publication No. 2004/0184867 A1, which is included by reference herein.

Treatment agents can be particularly useful for surfaces that are strongly reflective or strongly absorbing of UV radiation, and for surfaces that are rough, pitted, or have cavities or crevices. UV light may not be able to reach directly or with sufficient dose into all regions of irregular surfaces to substantially denature, sanitize or disinfect without the additional boost offered by treatment agents. Treatment agents can improve the efficacy of UV treatment by enhancing or changing the UV absorption properties of surface materials. Treatment agents can also extend the duration of treatment benefits to control or prevent the growth, deposition, penetration, or adhesion of bacteria, virus, molds, spores, allergens, and other harmful contaminants. Treatment agents can also make a surface easier to clean on future occasions or can repel contaminants such as dust and airborne particles.

Treatment agents can include photocatalysts, such as TiO₂, that can enhance the effectiveness of the UV light. Photocatalysts can be used regularly when cleaning, or they can be used very infrequently. In one arrangement, photocatalysts are applied to a surface of interest and the photocatalysts adhere to the surface. Every time a UV flash tool passes over the surface, the photocatalysts are re-activated and aid in treating contaminants. In another arrangement surface materials, such as carpets, flooring and countertops are manufactured with photocatalysts impregnated within. Again, when a UV cleaning tool passes over the surface, the photocatalysts are re-activated and aid in treating contaminants. For example, a process has been described wherein light-activated antimicrobial agents have been grafted onto the surface of fibers (Michielsen, Stephen, “Surface Modification of Fibers Via Graft-Site Amplifying Polymers,” International Nonwovens Journal, 12 (3), 41-44 (2003)). The effectiveness of the antimicrobial fibers increased as the intensity or time of light exposure increased.

UV-sensitive colorants change color when exposed to sanitizing UV light, thus confirming UV exposure. The color returns to normal within a few minutes. UV-sensitive colorants can be incorporated into surfaces or added during cleaning.

Many allergens and other contaminants have a natural charge and bond themselves electrostatically to surfaces, such as carpet fibers. In one embodiment treatment agents that can break or neutralize the electrostatic bonds that attach the contaminants to surfaces are used with the cleaning tool. Once the electrostatic bonds are broken, it is easier to vacuum or wipe away the contaminants. Examples of treatment agents that act electrostatically include baking soda and disodium octaborate tetrahydrate. In another embodiment, the cleaning tool includes a portion that has an electrostatic charge opposite the charge of a contaminant of interest. As the tool moves across a surface, the contaminant is attracted to the charged portion of the tool and becomes unbonded from the surface. The contaminant can be vacuumed or wiped away after it is pulled from the surface.

Other treatment agents include antifungal agents, allergen agglomerators, anti-odor agents, flame retardants, antimicrobial agents, and electrostatic agents. Allergen agglomerators can lift contaminants such as pollen, dead dust mites and their droppings off the surface of fabrics, carpets and curtains, and the like, and then coagulate them into larger blocks so that they can be removed easily. Antimicrobial agents may or may not be light activated. UV blocking agents can be used to protect carpets and other surfaces from UV damage from either the tool or from the sun.

In one embodiment, treatment agents can be generated onboard the combination tool. For example, an electrolysis cell can generate bleach or ClO₂ which can then be diluted according to the intended use—high concentration for stains, low concentration for additional disinfecting. In another example, ozone can be generated and then added to a liquid to form a treatment agent to sanitize and/or remove odor. Ozone can also be applied directly to the surface. Waste ozone can be filtered out of the exhaust stream from the tool.

This invention has been described herein in considerable detail to provide those skilled in the art with information relevant to apply the novel principles and to construct and use such specialized components as are required. However, it is to be understood that the invention can be carried out by different equipment, materials and devices, and that various modifications, both as to the equipment and operating procedures, can be accomplished without departing from the scope of the invention itself.

We claim:

1. A combination tool for cleaning and sanitizing a surface, comprising:
a cleaning device; and
a treatment unit adjacent the cleaning device, the treatment unit comprising at least one UV flash unit operative to emit pulses of ultraviolet radiation.

2. The tool of claim 1 wherein the cleaning device is adapted for dry cleaning.

3. The tool of claim 1 wherein the cleaning device is adapted for wet cleaning.

4. The tool of claim 1 further comprising a dispensing unit adjacent the cleaning device, the dispensing unit operative to dispense a treatment agent.

5. The tool of claim 1 wherein the treatment unit comprises a xenon flash lamp.

6. The tool of claim 1 wherein the treatment unit comprises a semiconductor ultraviolet optical source.

7. The tool of claim 1 further comprising a battery unit operative to provide electric power to the tool.

8. The tool of claim 7 wherein the battery unit is rechargeable.

9. The tool of claim 1 wherein the tool has a size less than about 1 cubic foot.

10. The tool of claim 1 wherein the tool has a size less than about 50 cubic inches.

11. The tool of claim 1 wherein the tool has a size less than about 10 cubic inches.

12. The tool of claim 1 further comprising a control system in communication with at least the treatment unit, the control system operative to control one or more features selected from the group consisting of on/off function, UV wavelength, UV intensity, UV flash duration, and time interval between flashes.

13. The tool of claim 12 wherein the control system further comprises a UV radiation safety interlock.

14. The tool of claim 12 wherein the control system further comprises a microcontroller or a logic device.

15. The tool of claim 12 further comprising a sensor adjacent the tool, the sensor operative to transmit information about the surface to the control system.

16. The tool of claim 15 wherein the information about the surface is selected from the group consisting of hardness, texture, moisture, roughness, reflectivity, and combinations thereof.

17. The tool of claim 12 further comprising a feedback mechanism operative both to gather information about cleaning device operating conditions and to transmit the information to the control system.

18. The tool of claim 1 further comprising a motive component in communication with the combination tool, the motive component operative to propel the tool.

19. The tool of claim 18 wherein the motive component operates robotically according to preprogrammed instructions.

20. The tool of claim 19 wherein the preprogrammed instructions direct the motive component to move the tool over the surface so that all accessible regions of the surface are covered by the tool at least once.

21. A tool for treating a surface, comprising:
a treatment unit comprising at least one UV flash unit operative to emit pulses of ultraviolet radiation;
a control system in communication with the treatment unit; and
a sensor adjacent the treatment unit, the sensor operative to transmit information about the surface to the control system.

22. The tool of claim 21 wherein the tool is cordless.

23. The tool of claim 21 wherein the tool is a hand-held.

24. The tool of claim 21 further comprising a cleaning device adjacent the treatment unit.

25. The tool of claim 21 further comprising a motive component in communication with the combination tool, the motive component operative to propel the tool robotically.

26. A combination tool for cleaning and sanitizing of surfaces, comprising:
a cleaning device;
at least one ultraviolet flash unit adjacent the cleaning device, the ultraviolet flash unit capable of emitting ultraviolet radiation having at least one wavelength between about 200 and 310 μm and for a flash duration less than about 1 second.

27. The tool of claim 26 wherein the ultraviolet flash unit is capable of emitting ultraviolet radiation having a flash duration less than about 100 milliseconds.

28. A combination tool for cleaning and sanitizing a surface, comprising:
means for cleaning the surface; and
means for emitting pulses of UV radiation onto the surface, the means for emitting pulses of UV radiation adjacent the means for cleaning.

29. A method of treating a surface, comprising the steps of:
providing a tool comprising:
a UV flash treatment unit;
a control system in communication with the treatment unit; and
a sensor adjacent the treatment unit, the sensor operative to transmit information about the surface to the control system;
positioning the tool proximate the surface;
activating the treatment unit; and
moving the tool along the surface.

30. The method of claim 29 wherein the tool further comprises a cleaning device adjacent the treatment unit.

31. The method of claim 29 wherein the UV flash unit comprises xenon gas.

32. The method of claim 29 wherein the UV flash unit comprises a semiconductor ultraviolet optical source.

33. The method of claim 29 wherein the step of moving the tool comprises the steps of:
providing a robotic motive component to the tool; and
activating the robotic motive component.

34. The method of claim 29 further comprising the step of applying a treatment agent to the surface before or during the step of moving the treatment unit along the surface.

35. The method of claim 34 wherein the treatment agent enhances the treating of the surface.

36. The method of claim 35 wherein the treatment agent is selected from the group consisting of photocatalysts, antifingal agents, allergen agglomerators, anti-odor agents, flame retardants, antimicrobial agents, and electrostatic agents.
